(19) United States
(12) Patent Application Publication
(10) Pub. No.: US 2003/0170296 A1
(43) Pub. Date: Sep. 11, 2003

(54) Transdermal drug delivery system

(76) Inventors: Amnon Sintov, Omer (IL); Raphael Gorodischer, Omer (IL)

Correspondence Address: FOLEY AND LARDNER, SUITE 500, 3000 K STREET NW, WASHINGTON, DC, 20007, US

(21) Appl. No.: 10/343,008

(22) PCT Filed: Jul. 10, 2001
(86) PCT No.: PCT/IL01/00630

(30) Foreign Application Priority Data
Jul. 27, 2000 (IL) 137559

(51) Int. Cl. A61K 9/70

(52) U.S. Cl. 424/449

(57) ABSTRACT

The invention provides a transdermal delivery system for analgesic, anti-pyretic and anti-inflammatory drugs comprising an analgesic, anti-pyretic or anti-inflammatory drug is combination with water-miscible tetraglycol and water for dissolving the drug in hydrogel form.

### BACKGROUND OF THE INVENTION

The present invention relates to a transdermal delivery system for analgesic, anti-pyretic and/or anti-inflammatory drugs. More particularly, the present invention relates to a transdermal delivery system for such drugs as acetaminophen, aspirin, capsaicin, diclofenac salts, or any analgesic-anti-pyretic agent that may be selected from the group consisting of non-steroidal anti-inflammatory drugs (NSAIDs) in a transdermal drug delivery system (TDDS).

The novel analgesic TDDS is preferably applied using a unilayer polymeric patch with adhesive margins, providing an effective and convenient mode of drug delivery. The formulation can be used to treat pain, inflammations, fever, or rheumatic diseases by local or systemic action.

A widely used NSAID is diclofenac. It is a highly effective nonsteroidal anti-inflammatory agent in the management of acute conditions affecting soft tissue, such as tendus, bursa and muscle. The topical application of diclofenac provides an alternative to the oral, rectal and parenteral dosage forms, and is particularly suitable for musculoskeletal pain and inflammation of well-defined areas near the body surface. The concentrations of the drug in the systemic blood circulation following a topical application are considerably lower than following other routes of administration. Therefore, this mode of administration is associated with reduced risk of systemic side effects, and in particular, gastrointestinal adverse reactions. A topical formulation, if properly designed to be locally effective (i.e., drug is highly absorbed into the peripheral blood in the site of action), may be beneficial in minimizing the inflammation process with a reduced risk to the patient. A topical composition of the commonly-used Voltaren® Emulgel®, containing 1.16% diclofenac diethylammonium corresponding to 1% diclofenac Na, is used for percutaneous treatment of localized form of non-articular rheumatism and inflammations. Diclofenac compositions for topical application based on oil-water emulsion and also contain gel formers and lower alkanol (“emulgel”) are described in Ciba's U.S. Pat. No. 4,917,886 (Apr. 17, 1990) or Israeli patent IL69925 (Apr. 29, 1988).

Diclofenac is soluble in aqueous solutions as ionized salts thus the penetration of the drug through the skin is dependent upon its partition into the lipophilic phase of the Emulgel®. An effective transdermal drug delivery system of diclofenac can be achieved according to the art of the present invention by formulating an aqueous-system containing the amphiphilic TG (tetraglycol) and the stabilizer GP (guar-based polymer), which are capable of carrying the drug quantitatively through the lipophilic skin. It has been discovered in the present invention that incorporating diclofenac sodium into formulations containing TG in a GP-based hydrogel patches enhanced drug penetration through the skin, while reduced drug accumulation in the skin tissue, thus avoiding a possible pathological damage and/or irritation to the skin.

Acetaminophen (paracetamol, APAP) is one of the most widely used medications and particularly in infants and in children. Its antipyretic and analgesic efficacy has been established in many placebo-controlled clinical trials in adults and in children. Oral administration of APAP is the most common route for this drug in the populations of all ages. However, the oral route of APAP administration may not be practical in infants and children with gastroenteritis (due to vomiting and diarrhea) and in sick young patients who often refuse to take the full dose of the medication orally (due to taste or other reasons). Therefore, an alternative route of administration may be most useful for pediatric use.

Following gastrointestinal absorption of therapeutic doses APAP reaches a peak in plasma within 30 to 60 minutes. It is uniformly distributed throughout most body fluids and undergoes extensive liver metabolism: at therapeutic doses 90-100% of the drug may be recovered in the urine after conjugation with glucuronic acid (about 60%), sulfuric acid (about 35%) and cysteine (about 2%); and unchanged APAP (about 2%); hydroxylated and deacetylated metabolites may also be found in limited extent. Newborn infants have a limited ability to conjugate APAP with glucuronide, but this is compensated by a well-developed sulfation pathway. A small proportion of APAP undergoes N-hydroxylation by cytochrome P-450 to form the highly reactive intermediate N-acetyl-benzoquinoneimine, which normally reacts with sulfhydryl groups in glutathione; however after toxic doses hepatic glutathione is depleted and this intermediate metabolite reacts with sulfhydryl groups of hepatic proteins leading to hepatic necrosis. APAP kinetic constants are as follows: Volume of distribution 0.95 L/kg, binding to plasma proteins 20%, clearance 0.3 L/kg/h and plasma elimination half life about 2 hours (similar in children and adults) and effective plasma concentrations 10-20 mcg/ml.

In therapeutic doses APAP is well tolerated; rare adverse reactions include allergic rashes, thrombocytopenia, neutropenia, pancytopenia; nephrotoxicity may be associated with chronic abuse of APAP. There is evidence that children have a greater capacity to metabolize APAP by nontoxic pathways; thus, weight-adjusted doses and serum concentrations that are associated with severe (even fatal) hepatotoxicity in young adults produce much less hepatocellular damage in preschool children following acute APAP overdose.

Usual APAP doses are 325-1000 mg/dose and up to 4000 mg daily in adults, and 10-15 mg/kg/dose every 4-6 hours up to five times/day for not more than 10 days in children an infants, or 1.5 g/square meter body surface area/day, in divided doses. APAP is available for oral administration in liquid and tablet forms, and also for rectal administration (although absorption by this route is variable). An oral sustained-released preparation of APAP has been marketed recently in the Unites States for pediatric use. However, these routes of administration may not be practical in infants and children with gastroenteritis (due to vomiting and diarrhea) and in sick young patients who often refuse to take the full dose of the medication orally (due to taste or other reasons). Therefore, an alternative route of administration may be most useful for pediatric use. No published information is available on the transdermal administration of APAP.

In principle, transdermal administration of medications has several advantages: elimination of variations in plasma concentration after gastrointestinal absorption, elimination of hepatic first pass metabolism, and avoidance of gastrointestinal intolerance. The dermal administration of NSAIDs may produce less gastrointestinal (GI) adverse reactions as compared with the oral route, as it is assumed that some of the GI adverse effects are due to the local action of the drug (e.g., in stomach). The transdermal route of administration may be of particular significance in infants and in children because of their greater surface area to weight ratio. The epidermis of the full term neonate (but not that of the premature infant) is well developed and similar to that of an older child or adult. But the thinner skin with relatively rich blood supply of the infant and child may affect the pharmacokinetics of drugs administered by transdermal delivery systems, this has obvious therapeutic advantages, but it may have also toxic significance (the majority of cases of percutaneous drug toxicity have occurred in infants: aniline dye, hexachlorophene, iodine, and alcohol poisoning). Transdermal drug absorption can significantly alter drug kinetics depending on a number of factors, such as site of application, thickness and integrity of the stratum corneum epidermis, size of the molecule, permeability of the membrane of the transdermal drug delivery system, state of skin hydration, pH of the drug, drug metabolism by skin flora, lipid solubility, depot of drug in skin, alterations of blood flow in the skin by additives and body temperature.

While topical drug delivery systems have been used for centuries for the treatment of local skin disorders, the use of the skin as a route for systemic drug delivery is of relatively recent origin. Transdermal administration of drugs has been established in adults in relation to nitroglycerine, estrogens, scopolamine, and fentanyl. Although lacking adequate pediatric studies, scopolamine and fentanyl are often used in pediatric patients by this route. There are data on the pharmacokinetics and clinical use of transdermal administration of theophylline in human neonates: after a single application to the skin an hydrogel disc system resulted in therapeutic concentrations of theophylline for up to 3 days in neonates with apnea.

### BRIEF DESCRIPTION OF THE INVENTION

According to the present invention there is now provided a transdermal delivery system for analgesic, anti-pyretic and anti-inflammatory drugs comprising an analgesic, anti-pyretic or anti-inflammatory drug in combination with water-miscible tetraglycol and water for dissolving said drug in hydrogel form.

In the practice of this invention topical analgesics-antipyretics such as APAP or diclofenac salts at concentrations from 0.1% to 80% by weight are incorporated into pharmaceutically acceptable carriers such as solutions, emulsion, gels, discs or patches. The resulting formulations can be re-applied several times daily to the skin of patients with muscuskeletal disorders, fever, or any type of pain. The hydrogel medium contains TG at concentrations ranged from 0.5% to 99% by weight, and an ionized polymer at concentrations ranged from 0% to 30% by weight.

Most NSAIDs and other topical drugs are practically insoluble in water or slightly soluble even in their ionized form. Therefore, dissolution of active agents in topical and transdermal preparations usually requires incorporation of an alcohol. However, the use of solvents like alcohols in topical preparations may lead to precipitation of the drug on the skin upon evaporation of the solvent once spread over the skin area. In situations in which the application area is occluded, such as in transdermal patches, alcohol presence may cause a skin irritation and inflammatory conditions.

The present invention obviates this problem since it enables the preparation of transdermal delivery systems for drugs usually requiring alcohol for the dissolution thereof, utilizing water-miscible tetraglycol instead of the standard alcohols used heretofor.

Thus, the present invention also provides a transdermal delivery system for an alcohol-miscible drug comprising an alcohol-miscible drug in combination with water-miscible tetraglycol and water for dissolving said drug in hydrogel form.

Another aspect of the present invention comprises the method of alleviating inflammation, fever and pain in humans and lower animals by a unique transdermal delivery system of a safe and effective amount of analgesic-antipyretic agent. This method includes preparation of ‘easy-to-make’ patches containing the drug, tetraglycol and other ingredients, which adhere spontaneously to the skin surface. The manufacturing method of transdermal patches of the present invention is unique by the virtue of the guar-based polymer to solidify the drug-containing liquid within few minutes to a patch at any desired size, shape and thickness. The manufacturing of the composition can be designed in such a way that once the polymer is dispersed in a liquid mixture containing the drug, the mixture is molded to a patch—a process that takes few minutes at the best case to several hours at the worst. The obtained patch is self-adhesive to the skin surface, requiring only a covering sheet with adhesive margins to occlude the system from any kind of evaporation or contamination during treatment.

### DETAILED DESCRIPTION OF THE INVENTION

It should be defined, that by the term “comprising” as used in the present invention is meant that various other inactive ingredients, compatible drugs and medicaments can be employed in the compositions as long as the critical tetraglycol or ionized polymers are present in the compositions and are used in the manner disclosed.

All percentages herein are by weight unless otherwise specified.

As stated hereinbefore, the transdermal delivery systems of the present invention comprise an effective amount of:

(a) an antipyretic, analgesic, or anti-inflammatory drug, or combinations of such drugs; and

(b) the water-miscible tetraglycol (TG), which can be mixed with any portion of water.

In addition, the transdermal systems of the present invention preferably include further components as follows:

(c) in the case where the composition according to the invention is a gel, soft or hard patch, stabilizers or shape-forming agents are selected from the group consisting of ionized polymers such as cationized guar gum, cellulose derivatives, acrylic polymers, polysaccharides, lipids, proteins, and polyhydroxy compounds. The average molecular weight of these polymers can vary from 5,000 to 500,000 daltons. The preferred polymer for the transdermal patch of the present invention is hydroxypropyl guar hydroxypropyltrimonium chloride (guar-based polymer, GP);

(d) in case that the composition is an emulsion, the oil phase comprises at least one ester selected from the group consisting of monoglycerides, diglycerides and triglycerides of monocarboxylic acids selected from the group consisting of saturated monocarboxylic acids and monocarboxylic acids containing ethylenic unsaturation. The emulsion is prepared by using pharmaceutically acceptable emulsifiers containing at least one esterified carboxylic group in its structure.

Further preferred components include:

(e) poly- or oligo-hyroxy compounds or their derivatives as co-solvents. These compounds can be selected from the group of polyalkylene glycols, poloxamers, and di- or tri-ethylene glycol ethyl ethers;

(f) skin penetration enhancers selected from nonionic surfactants consisting of sorbitan sesquioleate, cetostearyl alcohol, polysorbate 60, sorbitan monostearate, sorbitan monooleate, and a preferred combination of polyoxyethylene 23 lauryl alcohol and glyceryl mono/di-oleate;

(g) safe and effective preservatives such as parabens, benzyl alcohol and benzoic acid. pH adjusting agents such as triethanolamine, citric and lactic acid may also be included in the composition.

Preferred compositions according to the present invention are pharmaceutically accepted and easy-to-apply skin-adhesive systems containing NSAIDs as active ingredients. More particularly, these systems composed of tetraglycol (glycofurol, tetrahydrofurfuryl alcohol polyethyleneglycol ether) (TG) and an ionized polymer such as hydroxypropyl guar hydroxypropyltrimonium chloride (guar-based polymer, GP), which assist in dissolving or solubilizing the active materials in a hydrogel form, and facilitate their penetration through the lipophilic strata of the skin.

While the invention will now be described in connection with certain preferred embodiments in the following examples and with reference to the accompanying figures so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

In the drawings:

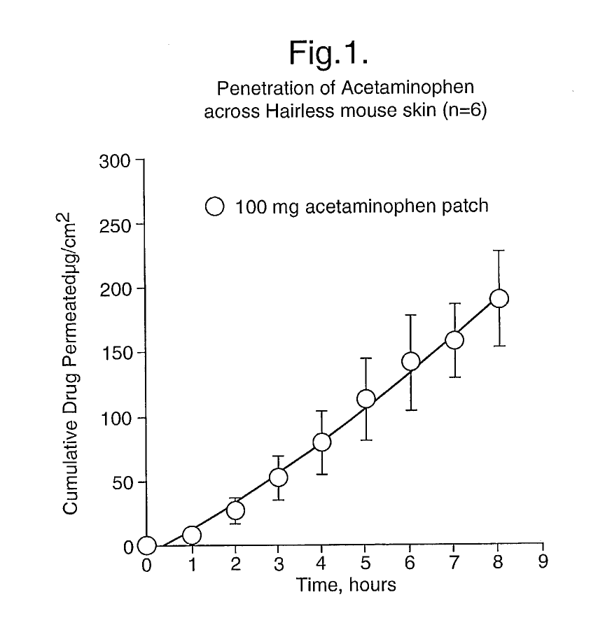

FIG. 1 is a graphical representation of the permeability of APAP through hairless mouse skin from a transdermal delivery system containing 100 mg per patch;

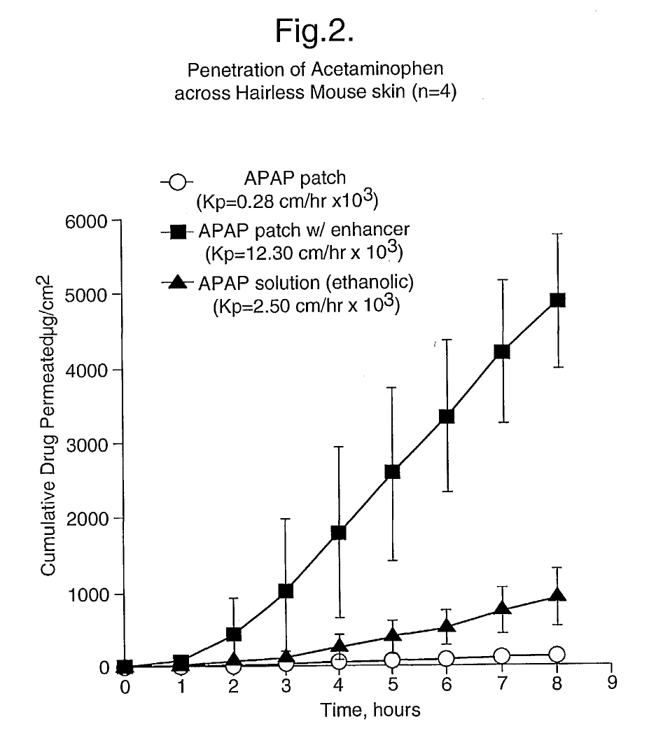

FIG. 2 is a graphical representation of the permeability of APAP through hairless mouse skin from a transdermal delivery system containing 100 mg per patch to which a penetration enhancer was added;

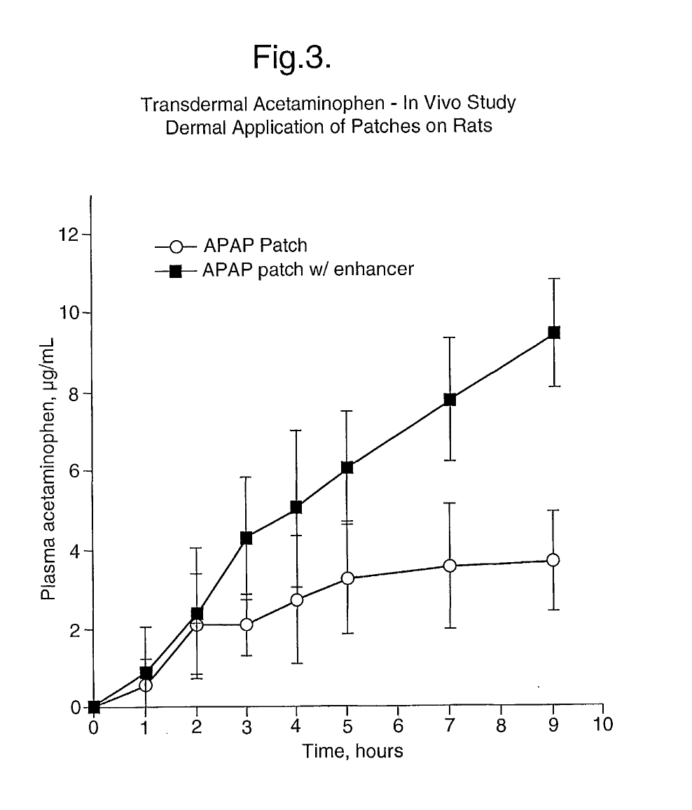

FIG. 3 is a graphical comparative representation of the permeability of APAP through hairless mouse skin from a transdermal delivery system containing 100 mg per patch with and without a penetration enhancer;

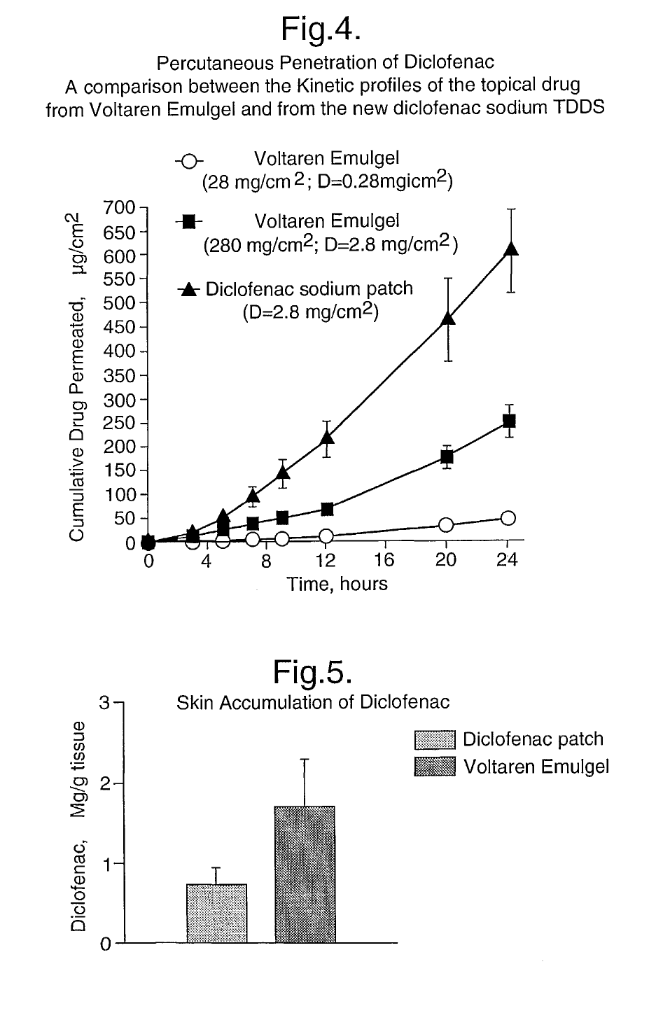

FIG. 4 is a comparative graphical representation of the permeability of diclofenac through hairless mouse skin from a transdermal delivery system containing 5 mg per patch, as compared to Voltaren Emulgel and;

FIG. 5 is a comparative graphical representation of the skin accumulation of diclofenac through hairless mouse skin from a transdermal delivery system according to the present invention, compared to that of Voltaren Emulgel.

### EXAMPLE 1

APAP-containing transdermal drug delivery (TDD) formulations

| | Formulation Code | | |
|---|---|---|---|
| Ingredients | PAC-3 | PAC-4 | AP/1-12 |
| Acetaminophen | 0.70 g | 0.70 g | 0.80 g |
| Tetraglycol (TG) | 1.4 ml | 1.4 ml | 1.74 g |
| Arlacel 186 | — | 0.70 g | 0.80 g |
| Distilled water | 1.7 ml | 1.7 ml | 0.3 ml/0.5 g of |
| | | | liquid |
| Jaguar C-162 | 50 mg/0.5 ml of | 50 mg/0.5 ml of | 0.50 g |
| | liquid | liquid | |

Jaguar C-162 = hydroxypropyl guar hydroxypropyltrimonium chloride (guar-based polymer, GP)
Arlacel 186 = glyceryl mono and dioleate

Preparation Procedure:

(PAC 3-4); acetaminophen (APAP) and TG were mixed together in a 10-ml vessel over a hot plate (80° C.) until complete dissolution of APAP was achieved. While still hot, Arlacel 186 and distilled water were added. The liquid was dispensed into 1-ml vials, 0.5 ml into each vial. 50 mg of Jaguar C-162 were added into each vial, mixed quickly, and the content was poured immediately into 1.5-cm diameter circular molds. After 5-10 minutes, the obtained patches were taken out and were ready for use on animal skin.

(AP/1-12): acetaminophen (APAP) and TG were mixed together in a 10-ml vessel over a hot plate (80° C.) with a magnetic bar, until a complete dissolution of APAP was achieved. While still hot (80° C.), Arlacel 186 and Jaguar C-162 were added. The liquid (under continuous stirring to prevent polymer sedimentation) was dispensed into 1.5-cm diameter circular molds, 0.5 g into each mold. Immediately after filling the molds, pre-heated distilled water was added, the liquid was mixed quickly in the mold for 30 seconds and was allowed to solidify.

### EXAMPLE 2

Diclofenac-containing transdermal drug delivery (TDD) formulations

| | Formulation Code | |
|---|---|---|
| Ingredients | DP/180400 | DP/140600 |
| Sodium | 0.70 g | 0.035 g |
| diclofenac | | |
| Tetraglycol (TG) | 1.52 g | 1.52 g |
| Arlacel 186 | 0.70 g | 0.70 g |
| Jaguar C-162 | 0.50 g | 0.50 g |
| Distilled water | 0.12 ml/0.375 ml | 0.12 ml/0.40 g |
| | of liquid | of liquid |

Jaguar C-162 = hydroxypropyl guar hydroxypropyltrimonium chloride (guar-based polymer, GP)
Arlacel 186 = glyceryl mono and dioleate.

Preparation Procedure:

TG and Arlacel 186 were mixed together in a 10-ml vessel over a hot plate (85° C.) with a magnetic bar. While still hot (80° C.), diclofenac sodium was dissolved, then Jaguar C-162 was added. The liquid (under continuous stirring to prevent polymer sedimentation) was dispensed into 1.5-cm diameter circular molds, 0.5 g (DP/140600) or 0.375 ml (488.6 mg) (DP/180400) into each mold. Immediately after filling the molds, pre-heated distilled water was added, the liquid was mixed quickly in the mold for 30 seconds and was left for up to 1 hour at room temperature before taking the patches out.

### COMPARATIVE EXAMPLE 3

In-Vitro Skin Permeation of APAP

Study methodology: The permeability of dermal APAP through hairless mouse skin was measured in vitro-with a Franz diffusion cell system (Crown Bioscientific, Inc., Clinton, N.J., USA). The diffusion area was 1.767 cm² (15 mm diameter orifice), and the receptor compartment volumes varied between 11.1 and 12.1 ml. The solutions on the receiver side were stirred by externally driven, Teflon-coated magnetic bars. Each set of experiments was performed with 6 diffusion cells. Sections of full-thickness hairless mouse (CD1, male, 6-7 weeks old, Weizmann Institute, Rehovot, Israel) abdominal skin were excised from the fresh carcasses of animals killed with ethyl ether. Subcutaneous fat was removed with a scalpel, and the skin sections were mounted in the diffusion cells. The skin was placed on the receiver chambers with the stratum corneum facing upwards, and then the donor chambers were clamped in place. The excess skin was trimmed off, and the receiver chamber, defined as the side facing the dermis, was filled with phosphate buffered saline (PBS, pH 7.4). After 30 minutes of skin washing at 37° C., the buffer was removed from the cells. APAP patches or alcoholic solutions were applied on the skin, and the receiver chambers were filled with phosphate buffer (4 mM, pH=7.4)—ethyl alcohol (analytical grade) (1:1). Samples (2 ml) were withdrawn from the receiver solution at predetermined time intervals, and the cells were replenished to their marked volumes with fresh buffer solution. Addition of solution to the receiver compartment was performed with great care to avoid trapping air beneath the dermis. Samples were taken into 1.5-m amber vials every hour for 8-hour period. The samples were kept at 4° C. until analyzed by HPLC. Aliquots of 20 □l from each vial were injected into the HPLC system, equipped with a prepacked C₁₈ column (Lichrosphere 100 CN, 5 mm, 250×4 mm). The detection of APAP was performed at 245 nm. The samples were chromatographed using an isocratic mobile phase consisting of water-methanol (3:1) at a flow rate of 0.5 ml/min. A calibration curve (peak area versus drug concentration) was constructed by running standard APAP solutions in ethanol-water for every series of chromatographed samples. Calibration curves were linear over the range 0.5-200 □g/ml. As a result of the sampling of large from the receiver solution—and the replacement of these amounts with equal volumes of buffer—the receiver solution was constantly being diluted. Taking this process into account, the cumulative drug permeation (Qₜ) was calculated from the following equation:

$${Q}_{t} = {V}_{r} {C}_{t} + {\sum }_{i = 0}^{t - 1} {V}_{S} {C}_{i}$$

where Cₜ is the drug concentration of the receiver solution at each sampling time, Cᵢ is the drug concentration of the i^{th} sample, and Vᵣ and Vₛ are the volumes of the receiver solution and the sample, respectively. Data were expressed as the cumulative APAP permeation per unit of skin surface area, Qₜ/S (S=1.767 cm²).

Results: FIG. 1 presents the permeability of APAP through hairless mouse skin from a transdermal delivery system containing 100 mg per patch. After 8 hours of application, the drug remaining (analyzed by HPLC) in the patches was 46.9±7.3% (Mean +S.D.) of the initial dose. Considering the relatively low proportion of drug penetrating into the receiver chamber, it is postulated that most of the APAP is accumulated in the skin and/or metabolized by skin enzymes.

When a penetration enhancer (Arlacel 186) was added into the formulation texture, the extent of APAP penetration increased by approximately 25 folds (see FIG. 2). The permeability coefficient (Kp; calculated according to Fick's equations) raised from 0.28×10⁻³ cm/hr (without an enhancer) to 12.3×10⁻³ cm/hr (with an enhancer). It should be noted that this dramatic elevation of APAP permeability from the patches cannot be even compared with the penetration observed upon dermal application of pure alcoholic solutions containing 125 mg of APAP (Kp=2.5×10⁻³ cm/hr) (FIG. 2).

### COMPARATIVE EXAMPLE 4

Pharmacokinetic Study of Transdermal APAP in Rats

Methodology: Anesthetized (15 mg/kg pentobarbital sodium i.p.) rats (Sprague-Dawley, 500-600 g), were placed on their back, the abdominal hair was trimmed off, and the skin was washed gently with distilled water. Anesthesia was maintained with 0.1 ml pentobarbital (15 mg/ml) along the experiment. APAP patches (1.8 cm²) were applied on the skin surface, covered and attached by an adhesive tape. Blood samples were taken from the tail vein into heparanized tubes at t=0, 1, 2, 3, 4, 5, 7 and 9 hours from the time of application.

After centrifugation, plasma were analyzed for APAP by EMIT kit (Serum Tox EIA Assay, Diagnostic Reagents, Inc.).

Results: FIG. 3 shows a study performed in 1.2 rats divided into two groups (n=6). The first group was treated with a regular 100 mg APAP patch and the other group was treated with 100 mg APAP in an enhancer-containing transdermal system. The obtained pharmacokinetic data support the in vitro results with hairless mouse skin, in which a significant increase in penetration occurred when using an enhancer. It has been concluded that the control the systemic penetration of APAP is possible, by simply tuning the enhancer's concentration, thus simulating a dose-response relationship. As the present invention is a topical composition having controlled release properties, the rate of penetration through the skin can be regulated, and much more rapid therapeutic concentrations may be achieved.

### COMPARATIVE EXAMPLE 5

In-Vitro Skin Permeation of Diclofenac

Study methodology: The permeability of dermal diclofenac through full-thickness hairless mouse skin was measured in vitro with a Franz diffusion cell system as described for the APAP permeation studies. Diclofenac patches or Voltaren Emulgel were applied on the skin, and the receiver chambers were filled with phosphate buffered saline (PBS, pH=7.4). The determination of diclofenac in the receiver fluid and in the skin was performed by HPLC. Aliquots of 20 □l from each sample were injected into the HPLC system, equipped with a prepacked C₁₈ column (Lichrosphere 60 RP-select B, 5 mm, 125×4 mm). The detection of diclofenac was carried out at 275 nm. The samples were chromatographed using an isocratic mobile phase consisting of dibasic sodium phosphate (0.008M, pH 2.5)—methanol (1:3) at a flow rate of 1 ml/min. Calibration curves were linear over the range 1-20 □g/ml.

Results: FIG. 4 shows the permeability of diclofenac through hairless mouse skin from a transdermal delivery system containing 5 mg per patch (n=6). For the purpose of comparison, 500 mg of Voltaren Emulgel containing 1% diclofenac (as sodium) were applied on the skin (1.8 cm² surface area, n=6). Since 500 mg emulgel (280 mg/cm²) is a relatively high application dose for semi-solid preparations, we also examined the permeability of diclofenac from regularly-spread 50 mg (28 mg/cm²) Voltaren Emulgel. As can be seen from the figure, 50 mg and even 500 mg of the commercial product delivered diclofenac through the skin at significantly lower quantities compared to the TDD patches. After 24 hours about 600 □g/cm² were diffused from the patches vs. 200 and 50 □g/cm² from 500 and 50 mg Emulgel, respectively. Since it is obviously not practical for the patient to spread 500 mg of emulgel over 1.8 cm² skin area, therefore, the patch of the present invention is capable in delivering 12-fold higher doses of diclofenac to the inflamed region than the regular application of Voltaren Emulgel. Inside the skin, the drug was accumulated 2- fold after Voltaren emulgel was applied than after the patch. This demonstrates that the TDDS of this invention has less potential for skin irritation or tissue damage, and increased potential for localized delivery of the drug to its target area.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A transdermal delivery system for analgesic, anti-pyretic and anti-inflammatory drugs comprising an analgesic, anti-pyretic or anti-inflammatory drug in combination with water-miscible tetraglycol and water for dissolving said drug in hydrogel form.

2. A transdermal delivery system according to claim 1, further comprising an ionized polymer.

3. A transdermal delivery system according to claim 2, wherein said ionized polymer is selected from the group consisting of catiolnized guar gum, cellulose derivatives, acrylic polymers, polysaccharides, lipids, proteins and polyhydroxy compounds.

4. A transdermal delivery system according to claim 2, wherein said ionized polymer is a guar-based polymer, which serves as a gelling agent for said composition.

5. A transdermal delivery system according to claim 3, wherein said guar-based polymer is hydroxypropyl guar hydroxypropyltrimonium chloride.

6. A transdermal delivery system according to claim 1, wherein said drug is selected from the group consisting of acetaminophen, aspirin, capsaicin, diclofenac salts and non-steroidal anti-inflammatory drugs.

7. A transdermal delivery system according to claim 5, wherein said non-steroidal anti-inflammatory drug is diclofenac.

8. A transdermal delivery system according to claim 1, wherein said transdermal delivery system is in the form of a hydrogel patch.

9. A transdermal delivery system according to claim 1, further comprising a skin penetration enhancer.

10. A transdermal delivery system according to claim 8, wherein said skin penetration enhancer is a non-ionic surfactant.

11. A transdermal delivery system according to claim 9, wherein said non-ionic surfactant is selected from the group consisting of sorbitan sesquioleate, cetostearyl alcohol, polysorbate 60, sorbitan monostearate, sorbitan monooleate, polyoxyethylene 23 lauryl alcohol, glyceryl mono/di-oleate and mixtures thereof.

12. A transdermal delivery system for an alcohol-miscible drug comprising an alcohol-miscible drug in combination with water-miscible tetraglycol and water for dissolving said drug in hydrogel form.

13. A method for alleviating inflammation, fever and pain in humans and in lower animals comprising providing a transdermal delivery system, including an effective amount of an analgesic, anti-pyretic agent, tetraglycol and water in the form of a hydrogel patch.
